(19) United States
(12) Patent Application Publication
(10) Pub. No.: US 2013/0183356 A1
(43) Pub. Date: Jul. 18, 2013

(54) IMMUNITY INDUCER

(71) Applicants: Jinyan Cheng, Tokyo (JP); Kouji Maruyama, Shizuoka (JP)

(72) Inventors: Jinyan Cheng, Tokyo (JP); Kouji Maruyama, Shizuoka (JP)

(21) Appl. No.: 13/812,000

(22) PCT Filed: Jul. 28, 2011
(86) PCT No.: PCT/JP2011/004305

§ 371 Date: Mar. 21, 2013

(30) Foreign Application Priority Data
Jul. 28, 2010 (JP) JP 2010-169055

(51) Int. Cl. A61K 33/42

(52) U.S. Cl. CPC A61K 33/42
USPC 424/400; 424/278.1

(57) ABSTRACT

The purpose is to provide an immunity-inducing agent which can be implanted or injected in vivo by a simple operation and is capable of activating the in vivo immunological actions without causing adverse side effects. An immunity-inducing agent comprising β-tricalcium phosphate (β-TCP) having a porosity of 50% or less is implanted or injected in vivo, particularly in the vicinity of a lesion area (non-lesion area) such as cancer. Lymphocytes such as T cells, B cells and NK cells or a dendritic cell can be induced in the vicinity of β-TCP, and a lymphadenoid tissue is formed in which lymphocytes are localized at a high density. Examples of the dosage form of the immunity-inducing agent include particles or granules having a particle or grain size of equal to or greater than 0.05 μm and less than 25 μm, as well as tablets or columellas.

### TECHNICAL FIELD

The present invention relates to an immunity-inducing agent comprising β-tricalcium phosphate (hereinafter also referred to as β-TCP) having a porosity of 50% or less. More specifically, the present invention relates to an immunity-inducing agent that induces lymphocytes and dendritic cells (hereinafter also referred to as “DC cells”) in its vicinity when transplanted or injected in vivo.

### BACKGROUND ART

Calcium phosphate compounds such as β-TCP have been brought into practical use as various products by virtue of its superior biocompatibility. For example, an artificial bone filler and the like is commercially available which comprises a high-purity β-TCP crystalline layer having a porosity of about 75% and consisting of 100-400 μm consecutive macropores and micropores and which has a quality of 2-3 MPa compressive strength, that is, a strength for easy workability at the surgery site (see e.g., Non-patent Document 1). Such β-TCP madreporites comprise an ideal property as a material for an artificial bone filler in that the β-TCP madreporites substitute for one's own bone, and this property is explained to be arisen from the absorbing action caused by two mechanisms, i.e., in vivo chemical dissolution and cellular phagocytosis (see e.g., Patent Document 1).

The present inventors have confirmed that malignant tumors can be suppressed by allowing β-TCP to directly contact with cancer with the use of a sheet for suppressing cancer cells in order to activate macrophages, wherein the sheet is made by coating the β-TCP madreporite powder having a particle size of 25-75 μm and a porosity of 75% or more onto the surface of a flexible sheet material (see e.g., Patent Document 2). In this invention, the accumulative action of lymphocytes and dendritic cells caused by β-TCP madreporite powder was not confirmed. Also in this invention, an invasive procedure of introducing the sheet was required so that sensitive procedures such as multiple dose administration and administration at varying concentrations were difficult to be carried out. Further, in a case of β-TCP madreporite powder, when the particle size is smaller than 25 μm, a cytosuppressive effect similar to when the particle size is 25-75 μm was observed at an initial stage, but there was a drawback that such cytosuppressive effect decreased over time.

### PRIOR ART DOCUMENTS

### Patent Documents

Patent Document 1: Japanese unexamined Patent Application Publication No. 2001-259016
Patent Document 2: Japanese unexamined Patent Application Publication No. 2010-126434

### Non-Patent Documents

Non-patent Document 1: Ceramics 43 (2008) No. 11, pages 967-988

### SUMMARY OF THE INVENTION

### Object to be Solved by the Invention

The object of the present invention is to provide an immunity-inducing agent that can be transplanted or injected in vivo by a simple operation and that are capable of activating immunological actions in vivo without causing adverse effects.

### Means to Solve the Object

The present inventors had varied the porosity of β-TCP to examine its immunity inducing action and found that when β-TCP having a porosity of 50% or less is transplanted or injected in vivo, lymphocytes such as B cells, T cells and natural killer cells (hereinafter also referred to as “NK cells”) and dendritic cells all involved in immunological actions are accumulated/induced in the vicinity region of β-TCP. Further, the present inventors have confirmed that a cellular structure similar to natural lymph node is constructed in a non-lesion area in the vicinity of the site of β-TCP transplantation. The present invention is thus completed.

The present invention thus relates to (1) an immunity-inducing agent comprising β-tricalcium phosphate (β-TCP) having a porosity of 50% or less; (2) the immunity-inducing agent according to (1), wherein the immunity-inducing agent is for in vivo implantation or injection; (3) the immunity-inducing agent according to (1) or (2), wherein the immunity-inducing agent induces a lymphocyte and a dendritic cell in its vicinity; (4) the immunity-inducing agent according to any one of (1) to (3), wherein the lymphocyte is one or more cell types selected from a T cell, a B cell and a NK cell; (5) the immunity-inducing agent according to any one of (1) to (4), wherein a dosage form of the immunity-inducing agent is a particle or a granule having a particle size of equal to or greater than 0.05 μm and smaller than 25 μm; (6) the immunity-inducing agent according to any one of (1) to (4), wherein a dosage form of the immunity-inducing agent is a tablet or a columella; and (7) a lymphadenoid tissue formed by the immunity-inducing agent according to any one of (1) to (6).

Other embodiments of the present invention include: a method for inducing an immune response comprising in vivo transplantation or injection of β-TCP having a porosity of 50% or less; a method for activating an immunological action comprising in vivo transplantation or injection of β-TCP having a porosity of 50% or less; a method for forming a lymphadenoid tissue by in vivo transplantation or injection of β-TCP having a porosity of 50% or less; a method of using β-TCP having a porosity of 50% or less as an immunity-inducing agent for in vivo transplantation or injection; a method of using β-TCP having a porosity of 50% or less as an agent for forming a lymphadenoid tissue for use in in vivo transplantation or injection; use of β-TCP having a porosity of 50% or less for preparing an immunity-inducing agent for use in in vivo transplantation or injection; and use of β-TCP having a porosity of 50% or less for preparing an agent for forming a lymphadenoid tissue for use in in vivo transplantation or injection.

### Effect of the Invention

According to the present invention, therapeutic effects can be provided on a lesion area through the activation of immunological actions by a simple operation of transplanting or injecting an immunity-inducing agent comprising β-TCP having a porosity of 50% or less in vivo, especially in the vicinity of a lesion area (non-lesion area) such as cancer. Also, prevention of the reoccurrence can be expected in a site in which a lesion area such as cancer has been excised. In addition, since what is transplanted or injected is a biocompatible β-TCP and what are induced are lymphocytes and dendritic cells originally present in the body of a patient, reactions other than the immune activation can be prevented from occurring in the body.

Further, when β-TCP particles or granules having a porosity of 50% or less and a particle size of 0.05 μm or greater and smaller than 25 μm are injected or transplanted, more lymphocytes and dendritic cells can be induced compared to when β-TCP particles or granules having a porosity of 50% or less and a particle size of 25 μm or greater are injected or transplanted. When a β-TCP tablet having a porosity of 50% or less is transplanted or injected, a long-lasting immunity inducing effect can be expected. When using β-TCP particles (granules) or a β-TCP columella having a porosity of 50% or less, multiple dose administration or local administration can be easily carried out with an injector, etc.

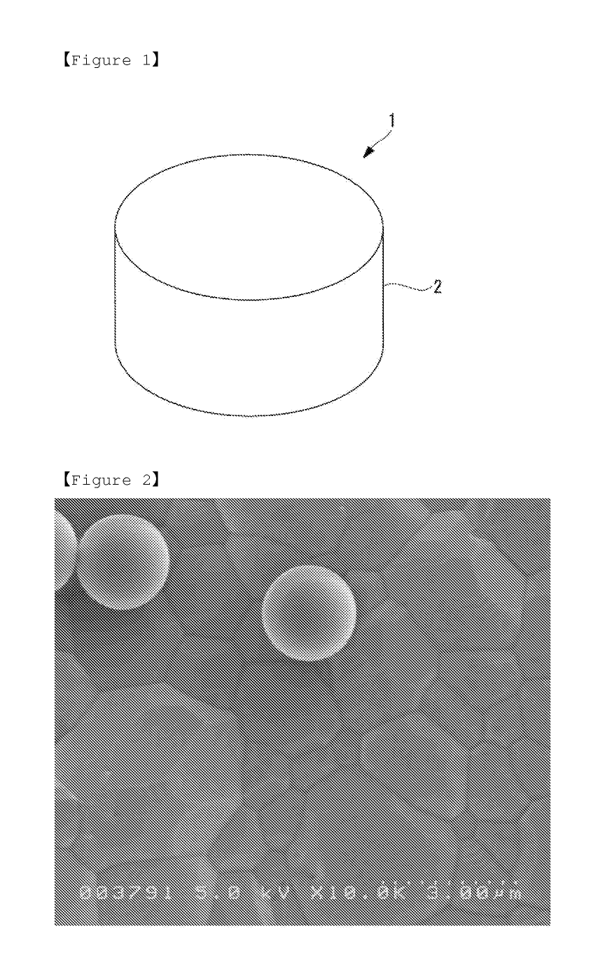

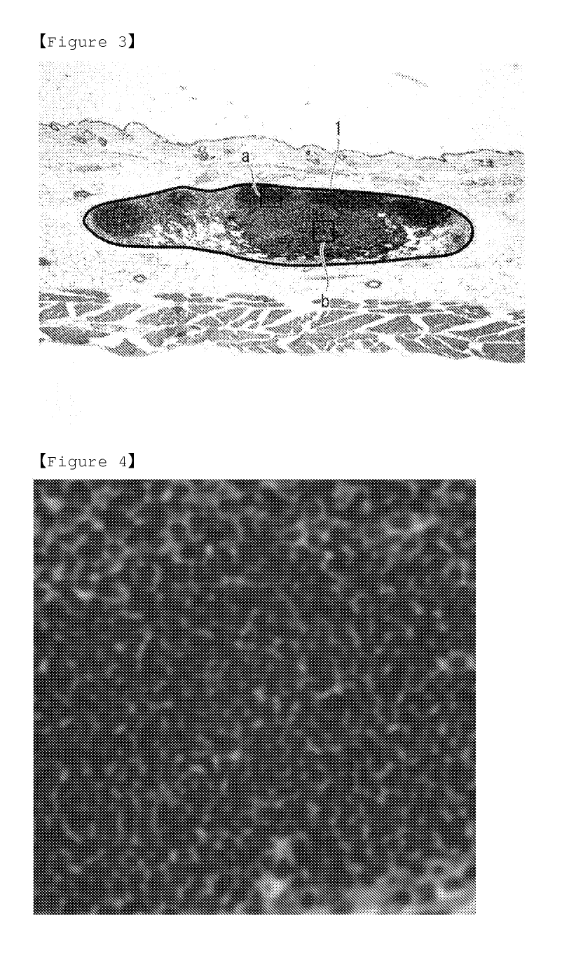

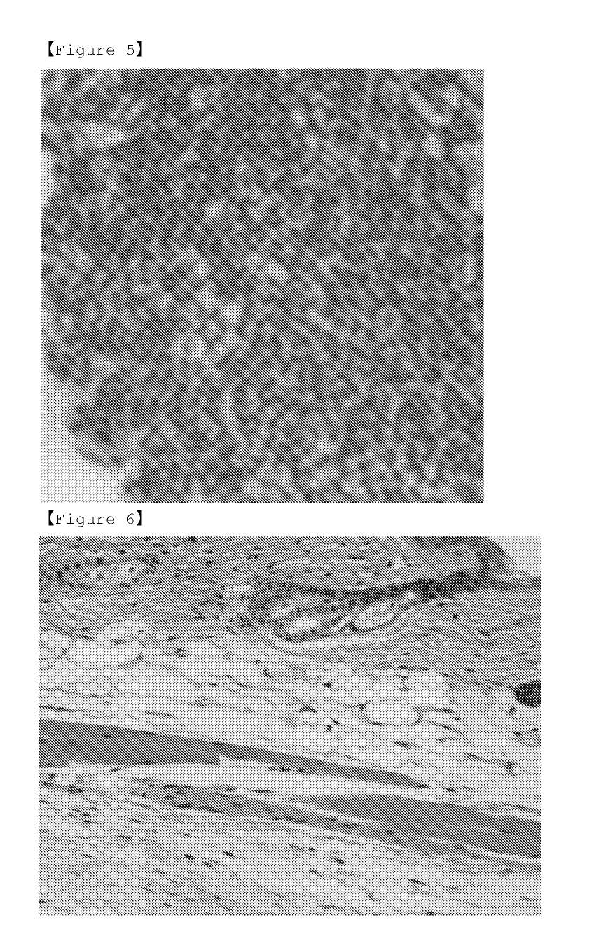

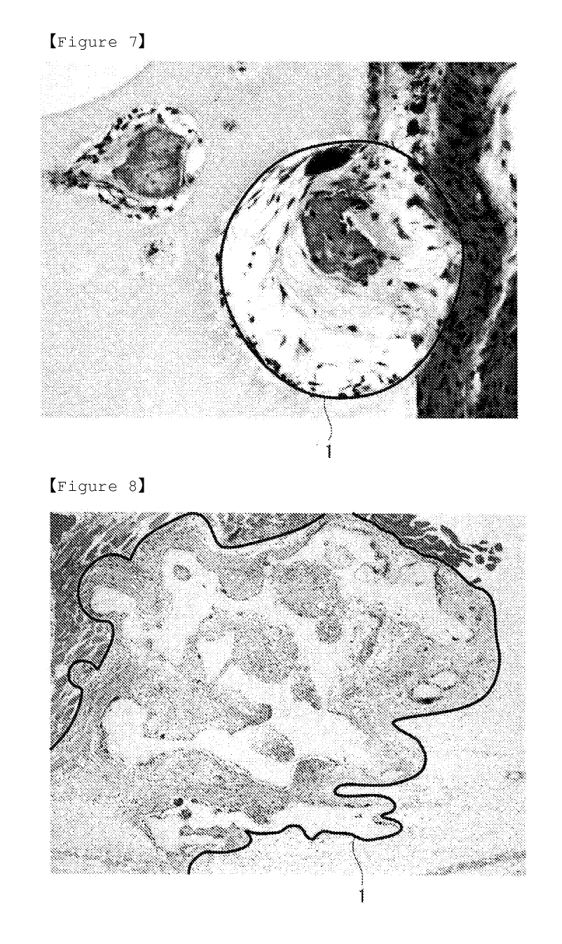

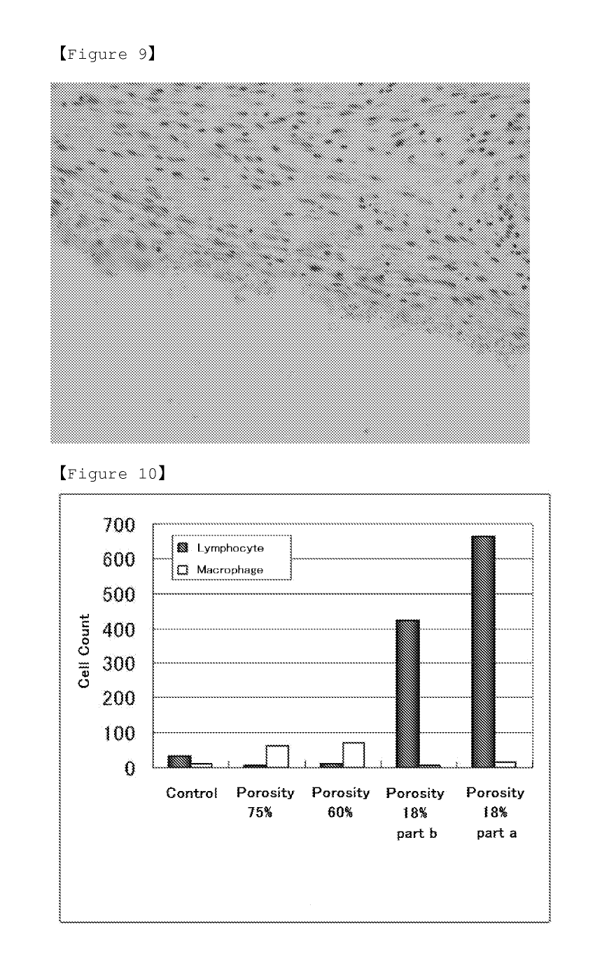

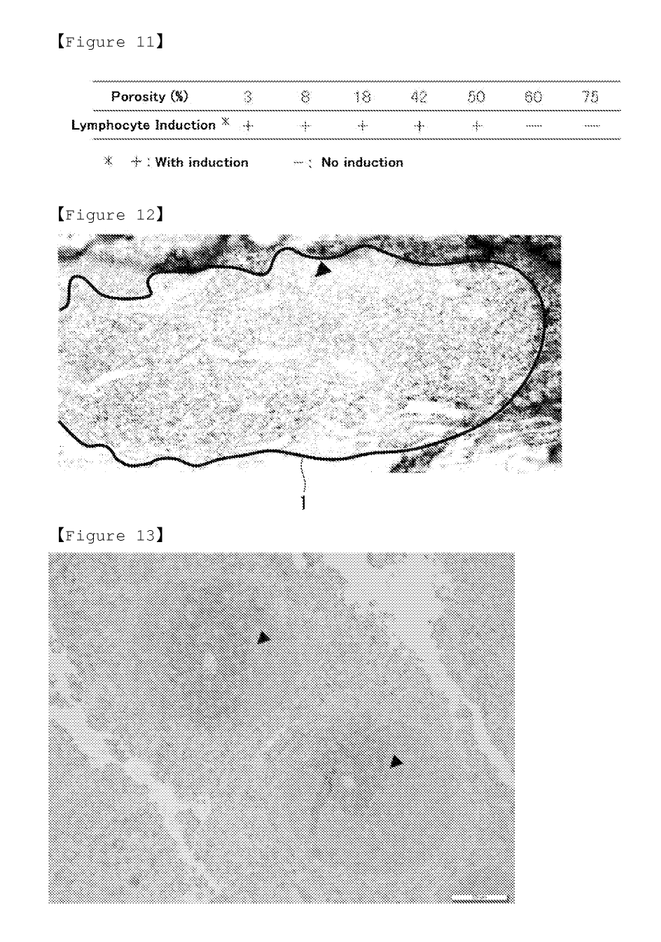

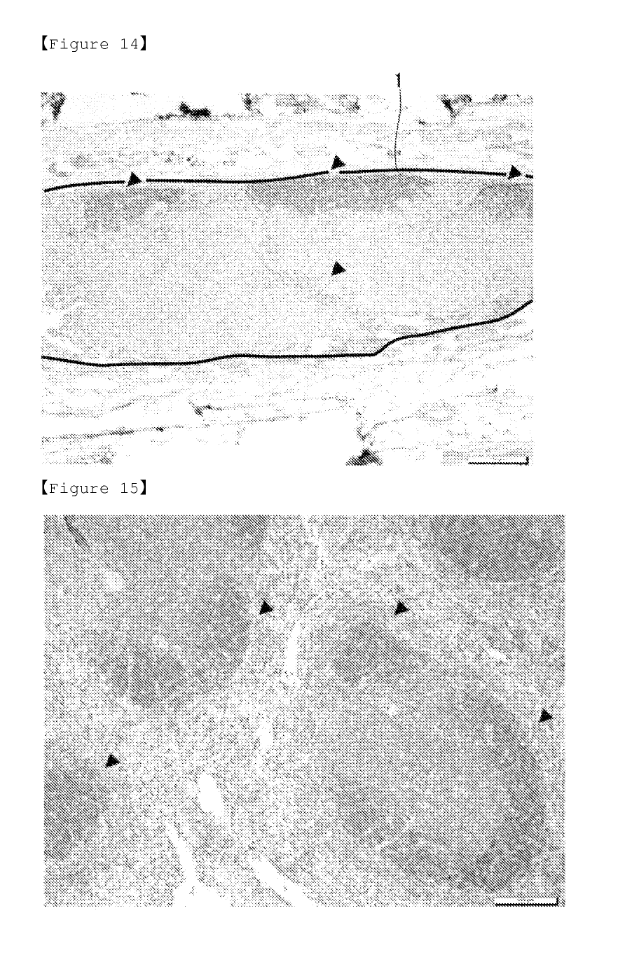

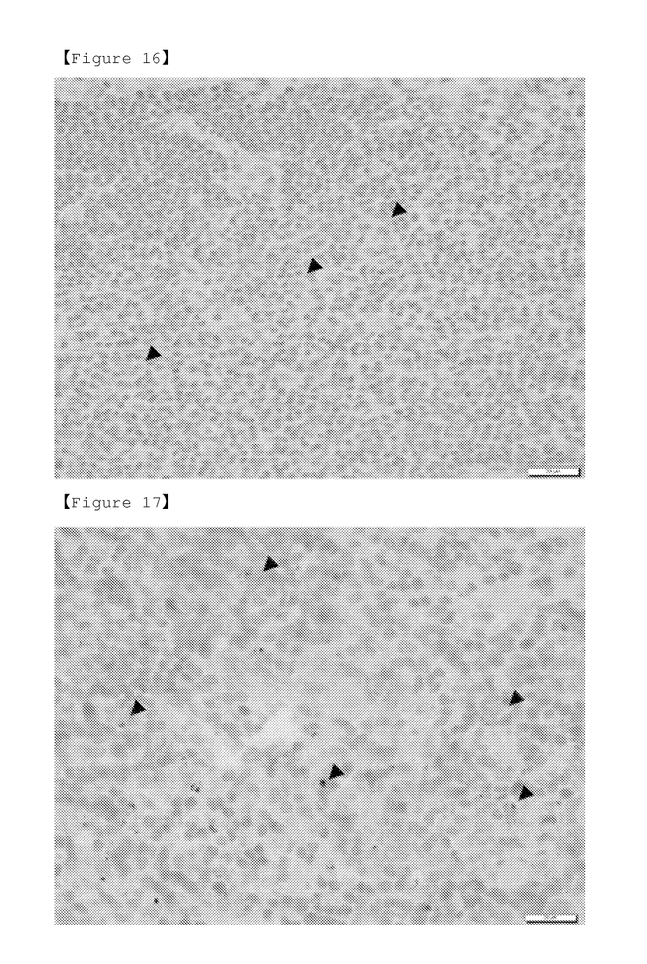

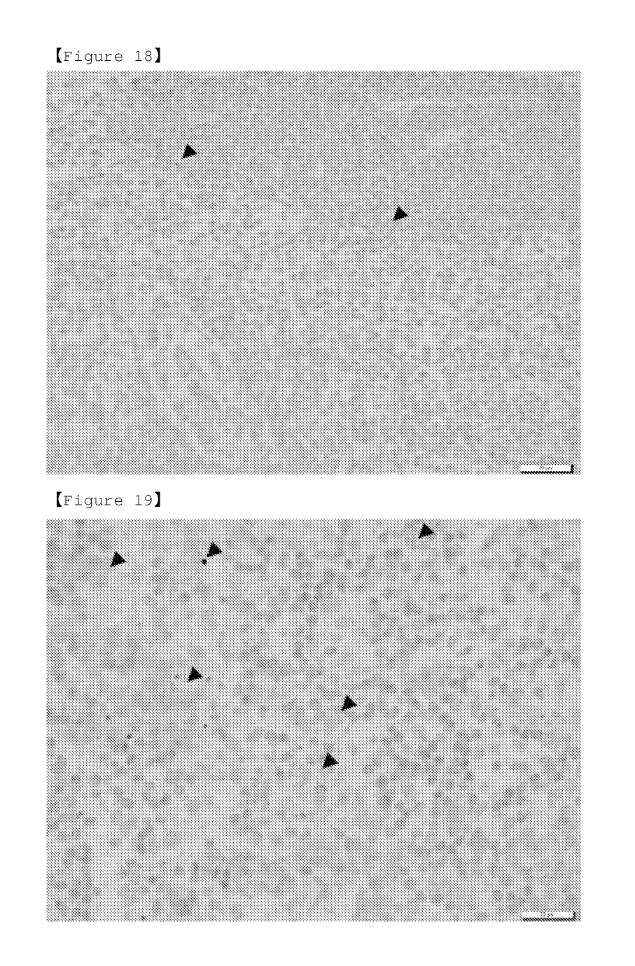

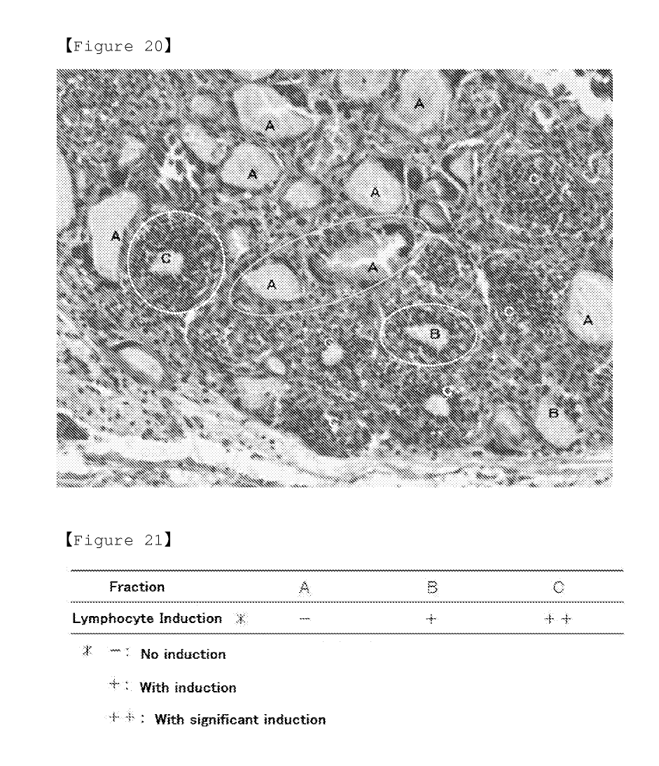

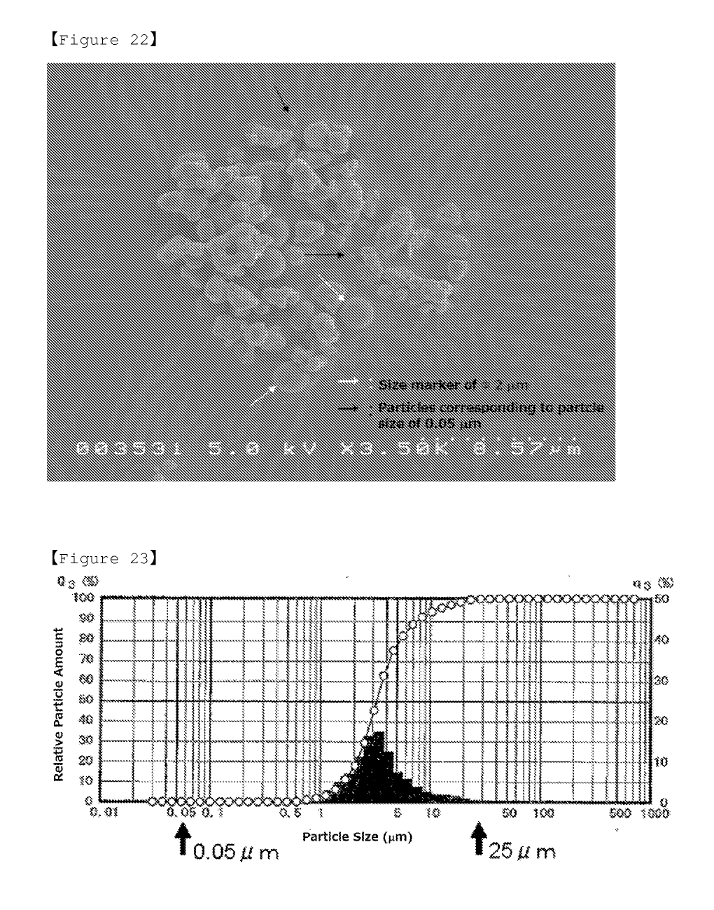

### BRIEF DESCRIPTION OF DRAWINGS

[FIG. 1] This figure shows an overall configuration diagram of a β-TCP tablet which is an embodiment of an immunity-inducing agent of the present invention.

[FIG. 2] This figure shows a SEM image of a torn surface of a β-TCP tablet having a porosity of 18%.

[FIG. 3] This figure shows a HE staining image of a dermal tissue transplanted with a β-TCP tablet having a porosity of 18%.

[FIG. 4] This figure shows a magnified image of the region ‘a’ in FIG. 3.

[FIG. 5] This figure shows a magnified image of the region ‘b’ in FIG. 3.

[FIG. 6] This figure shows a HE staining image of an untreated normal dermal tissue.

[FIG. 7] This figure shows a HE staining image of a dermal tissue transplanted with a β-TCP madreporite having a porosity of 60%.

[FIG. 8] This figure shows a HE staining image of a dermal tissue transplanted with a β-TCP madreporite having a porosity of 75%.

[FIG. 9] This figure shows a HE staining image of a dermal tissue transplanted with plastic.

[FIG. 10] This figure shows a graph showing the relationship between the type of a graft transplanted into a dermal tissue and the cell counts of lymphocytes and macrophages induced by the graft.

[FIG. 11] This figure shows a table showing the relationship between the porosity of β-TCP and the lymphocyte induction.

[FIG. 12] This figure shows an image (×10) of the transplantation site of β-TCP immunostained with a T cell-specific antibody.

[FIG. 13] This figure shows an image (×10) of a mouse splenic lymphatic tissue immunostained with a T cell-specific antibody.

[FIG. 14] This figure shows an image (×10) of the transplantation site of β-TCP immunostained with a B cell-specific antibody.

[FIG. 15] This figure shows an image (×10) of a mouse splenic lymphatic tissue immunostained with a B cell-specific antibody.

[FIG. 16] This figure shows an image (×40) of the transplantation site of β-TCP immunostained with a NK cell-specific antibody.

[FIG. 17] This figure shows an image (×40) of a mouse splenic lymphatic tissue immunostained with a NK cell-specific antibody.

[FIG. 18] This figure shows an image (×40) of the transplantation site of β-TCP immunostained with a DC cell-specific antibody.

[FIG. 19] This figure shows an image (×40) of a mouse splenic lymphatic tissue immunostained with a DC cell-specific antibody.

[FIG. 20] This figure shows a HE staining image of a dermal tissue transplanted with β-TCP powder.

[FIG. 21] This figure shows a table showing the relationship between the particle size of β-TCP powder and the cell induction.

[FIG. 22] This figure shows a SEM image of β-TCP powder fractions having particle size of 0.05-25 μm.

[FIG. 23] This figure shows a particle distribution diagram of β-TCP powder fractions having particle size of 0.05-25 μm.

### MODE OF CARRYING OUT THE INVENTION

An immunity-inducing agent of the present invention is not particularly limited as long as it is an immunity-inducing agent comprising β-tricalcium phosphate (β-TCP) having a porosity of 50% or less. Immunity induction herein means induction of immune response, that is, inducing an action caused by the immune system against a foreign substance (antigen). The β-TCP is one of the crystalline forms of the composition represented by Ca₃(PO₄)₂, and is a biocompatible compound that is stable at normal temperature. Further, the β-TCP is preferably a sintered compact.

A process of calculating the porosity of the (β-TCP is exemplified by a process using the formula: Porosity P (%)=(1-p′/p)×100, wherein p is a true density that has been measured for a β-TCP stone having a porosity of 0% (a sintered compact of β-TCP having the same composition as that of the sintered compact as a calculation target but not having crystalline pores) and p′ is an apparent density calculated from volume and weight of the sintered compact as a calculation target.

Examples of an immunity-inducing agent of the present invention include: β-TCP particles having a porosity of 50% or less; β-TCP granules (conjugates of plural particles) having a porosity of 50% or less; β-TCP tablets having a porosity of 50% or less; and β-TCP columellas having a porosity of 50% or less. These either may be consisted of β-TCP alone or may comprise β-TCP, as an effective component, and other components. Porosity of 50% or less for β-TCP is preferably 40% or less, more preferably 30% or less, and particularly preferably 20% or less.

The β-TCP particles or granules are administered in the vicinity of an in vivo lesion area (e.g., non-cancerous site) such as cancer in the form of powder, suspension (sol), viscous substance (gel), etc. Porosity P of such β-TCP particle or granule can be calculated with, for example, Accupyc 1330 series (SHIMADZU CORPORATION).

The β-TCP particles or granules can be produced by a method comprising, for example, weighing and mixing calcium hydrogen phosphate powder and calcium carbonate powder at a molar ratio of 1:1 to 1:3, preferably 1:1.5 to 1:2.5; adding pure water to the calcium hydrogen phosphate-calcium carbonate mixture to prepare a slurry; subjecting the prepared slurry to a wet-grinding treatment in a ball mill for about 24 hours to advance the reaction by a mechanochemical process; drying the wet-ground slurry at 70-90° C., preferably at 75-85° C.; grinding the solid substance, that was obtained after drying, to obtain β-TCP powder; temporarily sintering the obtained β-TCP powder for few hours at 700-800° C. to produce a temporarily sintered β-TCP powder; sieving the obtained temporarily sintered β-TCP powder to obtain fractions of each particle size; and obtaining factions having a porosity of 50% or less by measuring the porosity of each fraction. Specifically, first classifying those β-TCP as Fraction (A) that has passed a sieve having a sieve mesh of 105 μm but has not passed a sieve having a sieve mesh of 75 μm, then classifying those β-TCP as Fraction (B) that has passed a sieve having a sieve mesh of 75 μm but has not passed a sieve having a sieve mesh of 25 μm, and further classifying those β-TCP as Fraction (C) that has further passed a sieve having a sieve mesh of 25 μm. Under these classifications, β-TCP of Fraction (C) surely has porosity of 50% or less.

Examples of the particle size of the β-TCP particles or granules include equal to or greater than 0.05 μm and smaller than 500 μm, preferably smaller than 100 μm, more preferably smaller than 50 μm, further preferably smaller than 25 μm, and particularly preferably smaller than 15 μm. These particle sizes can be assessed by the fractionation using sieves as mentioned above, or based on the dimension of the β-TCP particles or granules as confirmed by comparing to the markers on a SEM image.

The dosage of the β-TCP particles or granules to be transplanted or injected will not be limited as long as the immunity inducing effect is exerted, but the dosage may be appropriately selected depending on the disease type, dimension of the lesion, weight of the patient, etc. Preferred examples of the dosage include 0.01 mg to 10 g, more preferably 0.1 mg to 10 g, and further preferably 1 mg to 1 g. Examples of the frequency of transplantation and/or injection include once every 1 to 6 weeks.

Examples of the form of the β-TCP tablet or columella include a columella such as a disk, cylinder, approximate cylinder, elliptic cylinder, and triangle- to heptagonal-columns. Size of the β-TCP tablet or columella may be appropriately determined by taking into account the type of lesion area, site of administration, dimension of the lesion, weight of the patient, etc. Examples of the diameter of the β-TCP tablet include 1.0-10.0 mm, preferably 3.0-7.0 mm and more preferably 4.0-6.0 mm, and the examples of the height include 0.5-4.0 mm, preferably 1.0-3.0 mm and more preferably 1.5-2.5 mm. Examples of the diameter of the β-TCP columellas include 0.1-2.0 mm, preferably 0.3-1.0 mm and more preferably 0.4-0.6 mm, and the examples of the length include 1.0-6.0 mm, preferably 2.0-5.0 mm and more preferably 3.0-4.0 mm.

Examples of the method for producing the β-TCP tablet or columella include a method that allows production of a monophase or almost monophase β-TCP tablet or columella having a porosity of 50% or less. Specifically, there is a method comprising: producing the temporarily sintered β-TCP powder in the above-mentioned manner; subjecting the obtained temporarily sintered β-TCP powder to a compression molding (tablet compression) to form the powder into a tablet or columella; sintering the formed temporarily sintered β-TCP tablet or columella for 2.5-3.5 hours, preferably for 3 hours at 450-650° C., preferably at 600° C., subsequently sintering for 0.5-1.5 hours, preferably for 1 hour, at 850-950° C., preferably at 900° C., and further sintering for 0.75-1.5 hour, preferably for 1 hour, at 1000-1300° C. to obtain a β-TCP tablet or columella as a sintered compact. Further, a β-TCP tablet or columella may be produced by sintering the temporarily sintered β-TCP powder to obtain powder as a sintered compact of β-TCP and subjecting the sintered compact of β-TCP to a compression molding (tablet compression). A β-TCP columella may also be produced by stamping out a β-TCP tablet in the shape of a column.

In the present invention, in vivo shall not be particularly limited if it is the site other than the bone and tooth, and the examples include subcutaneous site, intradermal site, intramuscular site, interperitoneal site, intrapleural site, and within the brain. The subcutaneous site is preferred. When an immunity-inducing agent of the present invention is transplanted and/or injected in vivo, lymphocytes such as T cells, B cells and NK cells as well as dendritic cells can be accumulated in the vicinity of the transplanted and/or injected immunity-inducing agent, and a lymphadenoid tissue can be constructed. A lymphadenoid tissue herein means a cell mass consisting of the accumulation of lymphocytes and dendritic cells.

Examples of the vicinity of an immunity-inducing agent where lymphocytes and dendritic cells are induced include a site which is located in the vicinity of a lesion area but is a non-lesion area, a remote site away from the lesion area, and a site from which the lesion area has been removed. Specific examples include no less than 1 mm and no more than 100 cm, preferably no more than 50 cm, more preferably no more than 5 cm and further preferably no more than 3 cm, for example 1.5-2.0 cm, distant from the lesion area or the site where a lesion has been removed. Lymphocytes and dendritic cells to be induced to the vicinity of the immunity-inducing agent are those lymphocytes and dendrtic cells originally present in the body of a patient so that reactions other than immune activation can be prevented from occurring in the body.

A lesion in the instant invention is not particularly limited as long as it is a lesion in other than the bone and tooth. Examples of the lesion include cancer, allergic immunity and infectious diseases, where cancer is preferred. Particularly, cancers in the skull tissue, lung, skin, mammary gland, soft tissue, bladder, stomach, liver, gallbladder, pancreas, head, neck, kidney, adrenal gland, prostate, large intestine, small intestine, esophagus, female genitalia (e.g., ovary and uterus), and thyroid gland are preferably exemplified.

Examples of the method for transplanting an immunity-inducing agent of the present invention include a method wherein the tablet mentioned above is provided at the site incised by surgical treatment, and a method wherein the particles or granules mentioned above are dispersed at the site incised by surgical treatment. Examples of the method for injecting an immunity-inducing agent of the present invention include a method wherein a suspension or the like of the particles or granules are directly injected in vivo via the minimum opening using an injector filled with the suspension or the like, and a method wherein the columella mentioned above is directly extruded and injected in vivo via the minimum opening using an injector equipped with an injection needle loading the columella.

As long as the effect of the present invention will not be diminished or inhibited, an immunity-inducing agent of the present invention may comprise, other than β-TCP, various pharmacologically acceptable pharmaceutical components such as an usual carrier, binder, stabilizer, excipient, diluents, pH buffer, disintegrant, solubilizer, solubilization agent and isotonic agent. These components may be injected by mixing with the particles or granules, or may be prepared by mixing the components with the particles or granules in advance and subjecting the mixture to a compression molding (tablet compression).

Hereinafter, the present invention will be described more specifically with reference to Examples. However, the technical scope of the present invention shall not be limited to these examples.

### EXAMPLE 1

(Preparation of β-TCP Tablet)

Calcium hydrogen phosphate powder and calcium carbonate powder were weighed and mixed at a molar ratio of 1:2. To the calcium hydrogen phosphate-calcium carbonate mixture, pure water was added at an appropriate ratio to prepare a slurry. The prepared slurry was subjected to a wet-grinding treatment in a ball mill for about 1 day. The ground slurry was dried for few hours at 80° C. The solid substance obtained after drying was ground to obtain β-TCP powder. The obtained β-TCP powder was temporarily sintered for few hours at 750° C. to produce a temporarily sintered β-TCP powder. The temporarily sintered β-TCP powder was accommodated in a container having the inner diameter of 5 mm and the height of 2 mm and compressed at a rotation speed of 100 rpm to form a tablet. The tablet was baked for 3 hours at 600° C., 1 hour at 900° C., and for further 1 hour at 1000-1300° C. The obtained β-TCP tablet was semi-translucent due to its partial crystalline structure and had a high hygroscopic property. FIG. 1 is the overall configuration diagram of a β-TCP tablet.

(Physical Property of β-TCP Tablet)

Porosity of the above β-TCP tablets was examined. The porosity was calculated with the formula: Porosity P (%)=(1-p′/p)×100, wherein p is a true density of a β-TCP stone having a porosity of 0% (a sintered compact having the same composition as that of a sintered compact as a calculation target but not having crystalline pores) and p′ is an apparent density calculated from volume and weight of the sintered compact as a calculation target. As a result, β-TCP tablets having porosities of 3%, 8%, 18%, 42%, 50%, 60% and 75% were obtained. FIG. 2 is an image of the observation of a torn surface of the β-TCP tablet having a porosity of 18% under a scanning electron microscope (SEM). The beads in the image are used as a size indicator and the bead size is 2 μm in diameter.

### EXAMPLE 2

(Examining the Effect of β-TCP Tablet on the Induction of Immune Cells)

Among the β-TCP tablets produced in the Example 1, a β-TCP tablet having a porosity of 18% was used to assess the effect of an immunity-inducing agent of the present application on the induction of immune cells by subcutaneously transplanting the tablet to 2 7-week-old mice. Specifically, the mice were anesthetized, incised the dorsal skin by about 10 mm and subcutaneously transplanted with a β-TCP tablet having a porosity of 18%. The mice were then bred for 2 weeks and the dermal tissue including the tablet-transplanted site was collected. The collected dermal tissue was fixed in formalin and embedded in paraffin to prepare thin sections to be used as pathological samples. The thin sections were subjected to HE (Hematoxylin-Eosine) staining and an optical microscopic observation.

FIGS. 3 to 5 show images of when a β-TCP tablet having a porosity of 18% was transplanted. FIGS. 4 and 5 are, respectively, magnified images of the marginal part ‘a’ or the central part ‘b’ in the site of transplantation in FIG. 3. FIG. 6 shows a HE-staining image of the thin section prepared from a dermal tissue which had not been transplanted with a β-TCP tablet as a negative control.

As a comparative experiment, the same protocol as in Example 3 was followed to conduct transplantation, breeding, preparation and staining of thin sections, and observation under a optical microscope, except that a β-TCP tablet having a porosity of 60%, a β-TCP tablet having a porosity of 75%, or a plastic section were used as the grafts instead of the β-TCP tablet having a porosity of 18%. FIG. 7, FIG. 8 and FIG. 9 show the results of transplantation using a β-TCP tablet having a porosity of 60%, a β-TCP tablet having a porosity of 75% and a plastic section, respectively. Further, counts of lymphocytes and macrophages induced by the grafts were compared in the HE-staining images of a dermal tissue to which each graft had been transplanted. The results are shown in FIG. 10.

(Result)

It was confirmed from FIGS. 3-5 that when a β-TCP tablet having a porosity of 18% was transplanted, many lymphocytes were distributed in the whole neighborhood of the tablet. And, it was confirmed that a lymphadenoid tissue where lymphocytes were localized at a high density was formed especially at the marginal part of the tablet. Specifically, as is known from FIG. 10, when the β-TCP tablet having a porosity of 18% was transplanted, about 120-650 fold counts of lymphocytes were present at the transplantation site as compared to those present in other grafts used in the comparative experiment, while macrophages were scarcely present. When β-TCP madreporites having 60% and 75% porosities were transplanted, osteoblasts were confirmed to be present as centering around the marginal part of the site of transplantation, while lymphocytes were scarcely confirmed. When plastic was transplanted, lymphocytes were confirmed to be present in a dispersed manner, but at very small counts.

The same analysis was conducted for β-TCP tablets having porosities of 3, 8, 18, 42, 50, 60 and 75% prepared in the Example 1, and the results are shown in FIG. 11. As a conclusion, lymphocytes were observed to have been induced at the porosity of 50% or less, but it was confirmed that β-TCP tablets having porosities of 60% and 75% scarcely induced any type of lymphocytes.

The above results confirmed that β-TCP tablets having a porosity of 50% or less effectively induced lymphocytes and that the β-TCP tablets have an immunity inducing action such that the immune function is activated by the induced lymphocytes.

### EXAMPLE 3

(Examination of Types and Distribution of Lymphocytes)

Types and distribution of the lymphocytes induced by the β-TCP tablets prepared in the Example 1 were examined by immunological staining. By the same protocol as in the Example 2, β-TCP tablets having a porosity of 18% was subcutaneously transplanted into mice, the mice was bred for 2 weeks after the transplantation, the site of transplantation was collected, thin sections were prepared that were immunostained with antibodies respectively specific to T cell, B cell, NK cell and DC cell. Further, thin sections of the lymph node collected from the mice that had been transplanted with an immunity-inducing agent were also immunostained for each of the cells. FIGS. 12-19 show the results of the optical microscopic observation of the vicinity of the site of transplantation of β-TCP having a porosity of 18%, and the non-transplanted splenic lymphatic tissue of the above mice.

FIGS. 12 and 13 respectively show staining of T cells at the vicinity of the site of transplantation and in the splenic lymphatic tissue. It can be seen that, in both regions, T cells are denser at the central part than at the marginal part.

FIGS. 14 and 15 respectively show staining of B cells at the vicinity of the site of transplantation and in the splenic lymphatic tissue. It can be seen that, in both regions, B cells are densely present at the marginal part (see arrows in FIGS. 14 and 15).

FIGS. 16 and 17 respectively show staining of NK cells at the vicinity of the site of transplantation and in the splenic lymphatic tissue. Cells pointed by the arrows are NK cells. Similarly in both regions, a few NK cells were confirmed to be present in a dispersed manner.

FIGS. 18 and 19 respectively show staining of dendritic cells at the vicinity of the site of transplantation and in the splenic lymphatic tissue. Cells pointed by the arrows are DC cells. Similarly in both regions, a few DC cells were confirmed to be present in a dispersed manner.

The above observation results of the immunological staining confirms that the β-TCP tablets produced in the Example 1 induced various types of lymphocytes such as T cells, B cells and NK cells and further induced dendritic cells as well. It was also confirmed that a cell mass structure similar to the normal lymph node was constructed after the tablets had been transplanted in vivo, wherein the cell mass structure had the similar distribution and density to those of the normal lymph node. These results suggest that a tablet comprising β-TCP of the present invention is a superior immunity-inducing agent that exerts immune function by inducing a lymphadenoid tissue similar to natural lymph node.

### EXAMPLE 4

(Preparation of β-TCP Particle or Granule)

The temporarily sintered β-TCP powder obtained in the Example 1 was sieved. Then, the particle size of those β-TCP that has passed a sieve having a sieve mesh of 105 μm but has not further passed a sieve having a sieve mesh of 75 μm was classified as “(A) 75 μm or greater and smaller than 105 μm”; the particle size of those β-TCP that has passed a sieve having a sieve mesh of 75 μm but has not further passed a sieve having a sieve mesh of 25 μm was classified as “(B) 25 μm or greater and smaller than 75 μm”; and the diameter of those β-TCP that has passed a sieve having a sieve mesh of 25 μm was classified as “(C) 0.05 μm or greater and smaller than 25 μm”, thereby obtaining three fractions (A) to (C).

(Porosity of β-TCP Particle or Granule)

Porosities of the three types of Fractions (A) to (C) were measured using a dry automatic density analyzer (Accupyc 1330, SHIMADZU CORPORATION), and it was confirmed that the porosity of Fraction (A) was 60-70%, the porosity of Fraction (B) was 40-50%, and the porosity of Fraction (C) was 0-30%.

(Examination of the Effect of β-TCP Particle or Granule on the Induction of Immune Cells)

Fractions (A) to (C) sorted as above were mixed in equal amounts and further suspended in PBS at 200 g/ml. Subsequently, the fractions were transplanted by subcutaneously injection of the suspension, and the thin sections were subjected to HE staining and an optical microscopic observation. FIG. 20 is an image of the observation result. FIG. 21 shows the relationship between the particle size of β-TCP powder and the cell induction.

In FIG. 20, A, B and C show β-TCP powder having porosities of 60-70%, 40-50% and 0-30%, respectively. It was confirmed that many macrophages were distributed in the neighborhood of A. On the other hand, lymphocytes were confirmed to be distributed in the neighborhood of B and to be localized at a considerably high density in the neighborhood of C. As can be seen from this, it was demonstrated that lymphocytes were more effectively induced especially for C. FIG. 22 is a SEM image of β-TCP particles having particle size of 0.05 ηm which is the lower limit of Fraction (C). Further, FIG. 23 shows a particle distribution of powder of Fraction (C).

### EXAMPLE 5

(Anti-Tumor Effect of β-TCP Tablet)

The anti-tumor effect in mice were examined using β-TCP disk tablets (5 mm in diameter and 2 mm in height) having a porosity of 0.1% to 12%. For control, a plastic carrier material of the same size was used which does not comprise any heterogeneous protein that may cause immunogenicity.

(Method)

A 6-week-old male nude mouse (BALB/c-nu/nu) was subcutaneously transplanted with 2.5×10⁶ human colon cancer cell COLO205 (purchased from ATCC). 5 to 7 days after the transplantation, size of the tumor formed in the mouse was measured to calculate the tumor volume. The tumor volume was calculated by the following formula.

Tumor volume=(major axis×minor axix²)/2 (mm³)

(Result)

A β-TCP tablet and a plastic carrier material as a control were respectively transplanted subcutaneously into the sites 15-20 mm apart from the formed tumor. About 1 month later, the tumor size was measured and the action of β-TCP on the tumor growth was observed. As a result, whereas the tumor volume in the control was 1400 mm³, the tumor volume in the β-TCP administered group was decreased to 1200 mm³.

### EXPLANATION OF CODES

1. Immunity-inducing agent
2. β-TCP

1-7. (canceled)

8. A method of using β-tricalcium phosphate (β-TCP) having a porosity of 50% or less as an immunity-inducing agent.

9. The method according to claim 8, wherein the immunity-inducing agent is for in vivo implantation or injection.

10. The method according to claim 8, wherein the immunity-inducing agent induces a lymphocyte and a dendritic cell in its vicinity.

11. The method according to claim 10, wherein the lymphocyte is one or more cell types selected from a T cell, a B cell and a NK cell.

12. The method according to claim 9, wherein the immunity-inducing agent induces a lymphocyte and a dendritic cell in its vicinity.

13. The method according to claim 12, wherein the lymphocyte is one or more cell types selected from a T cell, a B cell and a NK cell.

14. The method according to claim 8, wherein a dosage form of the immunity-inducing agent is a particle or a granule having a particle size of equal to or greater than 0.05 μm and smaller than 25 μm.

15. The method according to claim 9, wherein a dosage form of the immunity-inducing agent is a particle or a granule having a particle size of equal to or greater than 0.05 μm and smaller than 25 μm.

16. The method according to claim 10, wherein a dosage form of the immunity-inducing agent is a particle or a granule having a particle size of equal to or greater than 0.05 μm and smaller than 25 μm.

17. The method according to claim 11, wherein a dosage form of the immunity-inducing agent is a particle or a granule having a particle size of equal to or greater than 0.05 μm and smaller than 25 μm.

18. The method according to claim 12, wherein a dosage form of the immunity-inducing agent is a particle or a granule having a particle size of equal to or greater than 0.05 μm and smaller than 25 μm.

19. The method according to claim 13, wherein a dosage form of the immunity-inducing agent is a particle or a granule having a particle size of equal to or greater than 0.05 μm and smaller than 25 μm.

20. The method according to claim 8, wherein the dosage form of the immunity-inducing agent is a tablet or a columella.

21. The method according to claim 9, wherein the dosage form of the immunity-inducing agent is a tablet or a columella.

22. The method according to claim 10, wherein the dosage form of the immunity-inducing agent is a tablet or a columella.

23. The method according to claim 11, wherein the dosage form of the immunity-inducing agent is a tablet or a columella.

24. The method according to claim 12, wherein the dosage form of the immunity-inducing agent is a tablet or a columella.

25. The method according to claim 13, wherein the dosage form of the immunity-inducing agent is a tablet or a columella.

26. A method of forming a lymphadenoid tissue by the immunity-inducing agent according to claim 8.

27. A method of forming a lymphadenoid tissue by the immunity-inducing agent according to claim 9.
